(19) United States
(12) Patent Application Publication
(10) Pub. No.: US 2009/0064977 A1
(43) Pub. Date: Mar. 12, 2009

(54) METHOD TO DETERMINE AN ALCOHOL CONTENT

(75) Inventors: Oliver MIERSCH-WIEMERS, Stuttgart (DE); Georg Mallebrein, Korntal-Muenchingen (DE); Stephan Uhl, Markgroeningen (DE); Juergen Pfeiffer, Pforzheim (DE)

Correspondence Address: MERCHANT & GOULD PC, P.O. BOX 2903, MINNEAPOLIS, MN, 55402-0903, US

(73) Assignee: Robert Bosch GmbH, Stuttgart (DE)

(21) Appl. No.: 12/114,241

(22) Filed: May 2, 2008

(30) Foreign Application Priority Data
May 4, 2007 (DE) 10 2007 020 959.4

(51) Int. Cl. F02D 41/14; G01N 33/22

(52) U.S. Cl. 123/674; 73/23.31; 123/703

(57) ABSTRACT

The invention relates to a method to determine an alcohol content in the fuel of an internal combustion engine, in which the fuel is delivered to the individual cylinders of the internal combustion engine by way of a fuel supply via a fuel rail; and the exhaust gas of the internal combustion engine is discharged via an exhaust gas manifold of an exhaust gas system, wherein at least one exhaust gas probe is present, and in which current changes in the alcohol content of the fuel are evaluated from the signal flow of a lambda signal of the exhaust gas probe, and said signal results from a closed-loop lambda control.

With this method a change in the alcohol content of the fuel can be detected and an explicit condition for a change in fuel can be derived. Moreover with the method, fuel-mixture errors can be distinguished from a changed fuel composition after a filling of the tank (fueling).

### TECHNICAL FIELD

The invention relates to a method to determine an alcohol content in the fuel of an internal combustion engine, in which the fuel is delivered to the individual cylinders of the internal combustion engine by way of a fuel supply via a fuel distributor rail; and the exhaust gas of the internal combustion engine is discharged via an exhaust gas manifold of an exhaust gas system, wherein at least one exhaust gas probe is present.

### BACKGROUND

Present day internal combustion engines, which operate according to the principle of the gasoline engine, are operated with a fuel, which is obtained from crude oil and contains hydrocarbons. Regular gasoline and high octane gasoline are examples of such a fuel. As an alternative, alcohols obtained from plants, for example sugar cane, are increasingly being used as fuel.

A motor vehicle, which can use both types of fuel, is known as a fuel adaptable vehicle, or also as a “flexible fuel vehicle” or in short “flex-fuel vehicle” or flex-power vehicle. This type of vehicle can be operated with pure gasoline as well as with various similar fuels, such as, for example, ethanol, bioethanol or methanol-gas mixtures. Pure ethanol is denoted as E100 fuel. In contrast pure gasoline is denoted as E0 fuel. An arbitrary mixture with xx % ethanol is denoted as Exx. In Europe, Brazil and in the USA, typical fuels, which contain alcohol, are comprised of 75 to 85% alcohol (E75 respectively E85). The remaining 15 to 25% of the fuel is gasoline.

Because alcohol in contrast to gasoline has a significantly smaller stoichiometric ratio during combustion (9.0 instead of 14.7), an increase in the injected fuel quantity is required during the stoichiometric engine operation using alcohol. This problem is exacerbated by the fact that arbitrary mixtures of gasoline and alcohol occur in the fuel tank as a result of the fuels, with which the tank is filled. After filling the tank (fueling), information regarding said filling of the tank (fueling) must at the latest be available when the new fuel with different characteristics arrives at the fuel distributor rail, the so called fuel rail, respectively at the fuel-delivery control system. An exact knowledge of the alcohol content in the fuel significantly improves the drivability of the vehicle as well as the cold starting capability.

The engine management system of the flex-fuel vehicle must therefore adapt the engine function, especially the fuel injection function, respectively the fuel injection characteristic diagrams, to the corresponding fuel-mixture ratio. An accurate detection of the prevailing fuel-mixture ratio in the fuel tank is required for this purpose. In so doing, it is assumed that the mixture ratio in the fuel tank can only change if a quantity of fuel has been added. For this reason, the detection of a filling of the tank (fueling) assumes a primary role in a flex-fuel concept.

The state of the art is a detection, which detects the change in fuel level of a vehicle at rest (fuel sending unit, signal at terminal 15). A filling of the tank (fueling) is not detected when the engine is running. The disadvantage with this is that the measured fuel level very greatly depends on the degree to which the vehicle slants. An improved detection of a filling of the tank (fueling) is described in a parallel application of the applicant (female).

In regard to current flex-fuel systems, the separation of feed forward control errors of the closed-loop fuel-mixture control, which are caused, for example, by variable dispersions of the fuel injection valves or leakages in the intake manifold, from the use of fuel containing alcohol present a huge challenge. The possibility exists that mixture errors are mistakenly interpreted as an alcohol percentage in the fuel and vice-versa.

It is therefore the task of the invention to provide a method, which allows changes in the alcohol content of the fuel to be distinguished from feed forward control errors during the fuel-mixture preparation.

### SUMMARY

The task is thereby solved in that the current changes in the alcohol content of the fuel are assessed from a signal flow of a lambda signal of the exhaust gas sensor, which is the result of a closed-loop lambda control. With this method, a change in the alcohol content, for example an ethanol or methanol content, of the fuel can be detected and an explicit condition for a change in the fuel can be derived. Moreover, fuel-mixture errors can be distinguished from a changed fuel composition after a filling of the tank (fueling) with the method. This makes it possible for markets, which have strict governmental regulations with regard to emissions and diagnostics, for example Europe and the USA, to be able to offer a system capable of authorization.

A significantly higher quality with regard to the determination of the alcohol content, respectively with regard to the detection of a change in the fuel, can be achieved if cylinder specific lambda differences of the individual cylinders, which are the result of a closed-loop lambda control of the individual cylinders, are evaluated. Closed-loop lambda controls of this kind are known from the state of the art, for example from the German patent DE 19737840 C2 or from the German patent DE 10131179 A1. In an application of the applicant (female), which has not yet been published, a preferred method for the closed-loop lambda control of the individual cylinders in an internal combustion engine with multiple cylinders is described.

Provision is thereby made for the temporal changes in the lambda signal or the temporal sequence of the cylinder specific lambda differences of the individual cylinders to be evaluated. For this reason the change in fuel can assuredly be detected when a change in fuel is made with an altered alcohol content. It is particularly advantageous if a time point is determined when the fuel, which has just been put into the tank, arrives, for example, at the fuel injection valve of the first cylinder as a function of the volume of the fuel distributor rail up until the fuel injection valve of the first cylinder and the volume of the fuel line and while taking into account the prevailing engine operating points as a function of the engine rotational speed and the relative load, respectively the cylinder fillings.

It is particularly advantageous if the evaluation of the lambda signal or the evaluation of the temporal sequence of the cylinder specific lambda differences of the individual cylinders is started after the input of a fueling signal from a separate fueling detection. In the preferred variation, the fueling detection can evaluate the increase in the fuel level when the vehicle is at rest as well as when the vehicle is moving, as this is described in a parallel application of the applicant (female).

Provision is made in the preferred variation of the method for a condition for a change in fuel to be derived and/or the magnitude of the change of the alcohol content in the fuel to be determined from the evaluation of the signal flow of the lambda signal or from the temporal sequence of the cylinder specific lambda differences of the individual cylinders, which, for example, can result from the comparison of the lambda signals with, for example, threshold values, which are dependent on the operating point. Depending on the magnitude of the signal differences and their temporal occurrence, a change in fuel can then be detected and a corresponding signal can be set up for it; or a suggestion can be made about the alcohol content from the height of the signal changes. Furthermore, it is also thereby possible to detect a prevailing fuel-mixture error if, for example, the occurrence of the signal changes of the lambda signals and their signal height do not correspond to a signal pattern for a change in fuel. It is additionally advantageous to use the signal patterns which arise from the exhaust gas probes for diagnostic purposes in order, for example, to detect a malfunction of an exhaust gas probe.

If new adaptation values for a closed-loop lambda control are determined and stored after the determination of a change in the alcohol content, the altered stoichiometry of the new fuel can thereby correspondingly be taken into account during the fuel-mixture formation so that the fuel injection function, respectively the fuel injection characteristic diagrams, can be adapted to the new fuel-mixture ratio. In so doing, an optimal closed-loop lambda control is also possible when the fuel characteristics change.

Provision is made in a preferred variation of the method for a balancing to be implemented before the evaluation of the lambda signal or before the evaluation of the temporal sequence of the cylinder specific lambda differences of the individual cylinders, when a constant or homogenous fuel composition prevails in the fuel distributor rail of the internal combustion engine and/or when the fuel supply is flawless. In so doing, a standardization of the cylinder specific lambda signals can occur to compensate for tolerances and/or mistakes during the fuel-mixture formation, for example in the fuel injection valves. This standardized value can then be stored as a non-volatile adaptation value and is therefore provided to the closed-loop lambda control.

If prior to the evaluation procedure of the lambda signal, a balancing is implemented at a steady state operating point of the internal combustion engine, for example during idling of the internal combustion engine, the advantage arises, in that no dynamic changes interrupt the balancing process. This is especially the case when the internal combustion engine is idling.

If a mixing of the fuel is reduced in the fuel distributor rail by the introduction of one or several flow control valves in the fuel distributor rail between the cylinder groups or between the individual cylinders, variable dispersions and/or errors with the fuel injection valves of the individual cylinders can additionally be acquired with a higher degree of accuracy. Provision can thus be made, for example, in a four cylinder engine for a flow control valve to be disposed in the fuel distributor rail between the cylinders two and three. In this way the mixing of fuels with different characteristics can be largely prevented by, for example, pulsation or other flow effects, so that the evaluation procedure is not disrupted.

Provision can be made in an additional preferred variation for the volume of the fuel distributor rail to be enlarged by an ancillary volume for at least one cylinder or a cylinder group of the internal combustion engine, for example for the third and fourth cylinder of the four cylinder engine. This offers the advantage that the cylinders three and four can be operated longer with the still constant fuel-mixture ratio of the fuel before the tank was filled (fueling); and in so doing, a separation of the fuel with a changed composition arriving at the fuel rail is more possible. The ancillary volume of the fuel distributor rail as well as the volumetric proportion of the fuel distributor rail, which is directly connected to the ancillary volume, is thereby to be constructed in such a way that the complete volume of this section of the fuel distributor rail stands in relation to the current fuel consumption during balancing, for example to the fuel consumption when idling, in a manner which allows for a significant breakup of the temporally changing lambda signal.

Provision is made in a preferred application for the method to be employed with the previously described variations in internal combustion engines, which can be operated with a fuel with a varying alcohol content, as is the case with the flex-fuel vehicle (FFV) and those which have intake manifold fuel injection or direct gasoline injection.

### BRIEF DESCRIPTION OF THE DRAWINGS

The invention is explained in detail below using the examples of embodiment depicted in the figures. The following are shown:

FIG. 1 is an internal combustion engine as the technical environment of the method according to the invention; and

FIG. 2 is a flow diagram of the method in schematic depiction.

### DETAILED DESCRIPTION

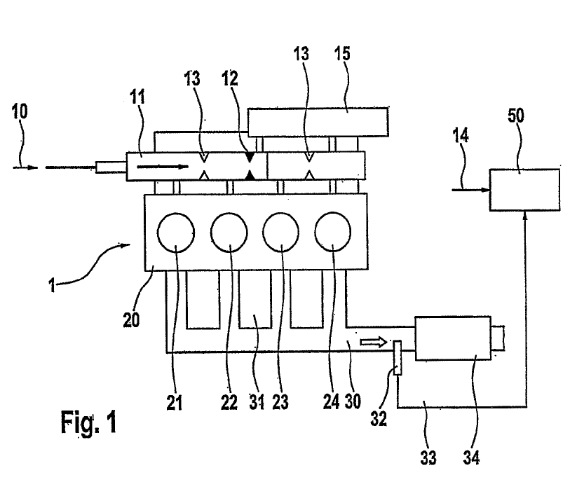

FIG. 1 shows in schematic depiction the technical environment, wherein the method according to the invention is used. An internal combustion engine 1 is depicted, which has an engine block 20, which in the example shown has four cylinders 21 through 24, at which the exhaust gas of these four cylinders 21 through 24 is collected in an exhaust gas system 30 in an exhaust gas manifold 31 and is discharged to an exhaust gas treatment system.

It is current practice, as can be recognized in FIG. 1, for provision to be made in the exhaust gas system 30 for at least one catalytic converter 34 to serve as the exhaust gas treatment system. At least one exhaust gas probe 32, with which a lambda signal can be determined, is disposed in each case in the exhaust gas duct in front of said catalytic converter(s) 34 in the direction of flow of the exhaust gas. As the output signal of the exhaust gas probes, the exhaust gas probe 32 delivers a lambda signal 33, which is provided to an evaluation unit 50. A fueling signal 14, which is derived from a separate, in this case unspecified fueling detection, can yet additionally be applied to the input side of the evaluation unit 50.

The fuel feed of the internal combustion engine 1 results by means of a fuel supply 10 (for example a fuel pump), which provides the fuel to a fuel distributor rail 11 (fuel rail). The fuel supply is designed as “returnless” in the application of the method according to the invention, i.e. the fuel distributor rail 11 has no return line to the fuel tank.

If a filling of the tank (fueling) now takes place with a fuel different from that found in the tank, the fuel with the altered characteristics will first reach the fuel distributor rail 11 and subsequently in succession cylinder one 21, then cylinder two 22 and after that cylinder three 23 and then finally cylinder four 24, which are supplied with fuel by the fuel distributor rail 11, after a period of time, which is dependent on the volume of the fuel supply and the operating point of the internal combustion engine. When the fuel with a changed composition, which also dependent on its composition has a different stoichiometry, arrives at the fuel distributor rail 11, a different lambda value then first regionally appears at cylinder one 21. The changes in the lambda value then follow with a time lag at the other cylinders 22 through 24. These changes can be evaluated from the lambda closed-loop control. In a preferred form of embodiment, the evaluation results from the cylinder specific lambda values, which result from an individual cylinder-closed-loop lambda control. These time lags can then, for example, be evaluated in the evaluation unit 50.

Between the first two cylinders 21, 22 and the cylinders three and four 23, 24, provision can optionally be made for a flow control valve 12 in the fuel distributor rail 11 as is depicted in FIG. 1. In this configuration, the firing order of the cylinders 21 through 24 must be taken into account. The flow control valve 12 can, for example, be designed as a perforated metal plate in the fuel distributor rail 11 and prevents a rapid mixing of the new fuel with the changed characteristics with the fuel still present in the fuel distributor rail 11 as a result of pulsation or other flow effects. The dispersion of the fuel with a changed composition ideally occurs gradually in the direction of flow in the fuel distributor rail 11. Provision can optionally be made for additional flow control valves 13 in the fuel distributor rail 11 between the first and second cylinder 21, 22 and additionally between the third and fourth cylinder 23, 24.

The development of an ancillary volume 15 at the fuel distributor rail 11 for a cylinder pair, in the example shown for cylinders three and four 23, 24, represents an optional structural extension of the embodiment.

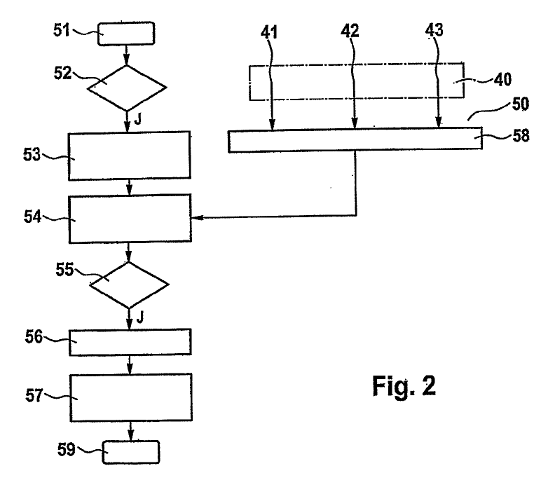

FIG. 2 shows a flow diagram of the method according to the invention as a component part of the evaluation unit 50.

The functionality according to the invention deals with a non-continuously running process. The activation first occurs after a start 51 if a filling of the vehicle's tank (fueling) has been detected with a fueling query 52. If this is the case, the evaluation system is activated in a manner, which assumes a change in the fuel composition situated in the tank of the vehicle has possibly taken place. In a calculation unit 53 the time point, at which the fuel most recently put into the tank arrives, for example, at the fuel injection valve of the first cylinder 21, is determined as a function of the volume of the fuel distributor rail 11 up until the fuel injection valve of the first cylinder 21 and the volume of the fuel line while taking into account the prevailing engine operating points as a function of the engine rotational speed and the relative load, respectively the filling of the cylinder, by means of integration of the quantity of fuel injected.

An evaluation of the lambda signal 33 of the exhaust gas sensor 32 within the exhaust gas manifold 31 takes place below in a lambda signal evaluation unit 54 when the fuel with a changed composition is expected to reach the fuel injection valves. This occurs while taking into account the cylinder firing order of the internal combustion engine 1. In a preferred variation of the method, a cylinder specific evaluation takes place with an individual cylinder-closed-loop lambda control.

A standardization of the lambda signal 33 was previously executed in a standardization unit 58 and was stored as a non-volatile adaptation value.

For the formation of these standardized values, a constant mixing ratio 43 in the tank and a flawless fuel supply 41 are assumed as starting conditions 40. It can additionally be advantageous to use a steady state operating point 42 as a reference for the standardization. In one variation, the idling state of the internal combustion engine 1 can, for example, be used for this purpose.

As soon as the lambda signal 33 of the exhaust gas manifold 31, respectively the lambda signals for the individual cylinders 21 through 24, exceeds a threshold, which can be established by means of a lambda signal query 55, the system detects a filling of the tank (fueling) with a fuel with changed characteristics. If this is the case, a condition of state for a detected “fuel with a changed composition in the system” is set up in an evaluation unit 56. In so doing, the cylinder firing order and the direction, in which new fuel flows through the system, must be taken into account.

As soon as the condition “fuel with a changed composition in the system” is present, a fixing of the adaptation values of the closed-loop lambda control occurs in a subsequent calculation and storage unit 57. The changes in the stoichiometry during the fuel-mixture formation, which now arise, are stored as a new alcohol value in the system and are used for the adaptation of the quantity of injected fuel.

The evaluation procedure only then proceeds to its termination 59 and can be restarted when a new fueling signal 14 emerges.

The method described can be implemented as a software and/or hardware solution and at least be a part of the overriding engine management system. The method can be employed in engines with intake manifold fuel injection (SRE) as well as in those with direct gasoline injection (BDE).

1. A method of determining an alcohol content in fuel, wherein the fuel is delivered to a plurality of individual cylinders of a combustion engine from a fuel supply via a fuel distributor rail, wherein an exhaust gas of the internal combustion engine is discharged via an exhaust gas manifold of an exhaust gas system having at least one exhaust gas probe, the method comprising:
evaluating current changes in the alcohol content of the fuel from a signal flow of a lambda signal of the at least one exhaust gas probe, wherein the lambda signal results from a closed-loop lambda control.

2. A method according to claim 1, further comprising evaluating a cylinder specific lambda difference, wherein cylinder specific lambda differences of the plurality of individual cylinders result from an individual cylinder closed-loop lambda control.

3. A method according to claim 2, further comprising evaluating a lambda signal temporal change or a temporal sequence of the cylinder specific lambda differences of the plurality of individual cylinders.

4. A method according to claim 3, wherein evaluating is started after an input of a fueling signal from a separate fueling detection.

5. A method according to claim 4, further comprising deriving a condition for a change in fuel and determines a magnitude of the change in the alcohol content of the fuel from the evaluation of the signal flow of the lambda signal or from the evaluation of the temporal sequence of the cylinder specific lambda differences of the plurality of individual cylinders.

6. A method according to claim 5, further comprising determining and storing one or more new adaptation values for the closed-loop lambda control after the determination of the change in the alcohol content.

7. A method according to claim 4, further comprising implementing a balancing procedure when a fuel composition in the fuel distributor rail is constant and homogenous or when the fuel supply is flawless, wherein the balancing procedure is implemented prior to the evaluation of the lambda signal or prior to the evaluation of the temporal sequence of the cylinder specific lambda differences of the plurality of individual cylinders.

8. A method according to claim 7, wherein the balancing procedure is implemented at a steady state operating point of the internal combustion engine.

9. A method according to claim 1, further comprising reducing a mixing of the fuel in the fuel distributor rail by an introduction of one or more flow control valves between a cylinder group or between the plurality of individual cylinders in the fuel distributor rail.

10. A method according to claim 9, further comprising reducing a volume of the fuel distributor rail by an ancillary volume for at least one of the plurality of individual cylinders or for a cylinder group of the internal combustion engine.
